(19) United States
(12) Patent Application Publication
(10) Pub. No.: US 2026/0041700 A1
(43) Pub. Date: Feb. 12, 2026

(54) IP-6 And Inositol Nerve Relaxing Transdermal Topical Cream, Gel, Or Ointment For Raynaud's Syndrome

(71) Applicants: John Vanderlinden, Coronado, CA (US); Kim Vanderlinden, Coronado, CA (US); Carla Janie Rosewall, Coronado, CA (US)

(72) Inventors: John Vanderlinden, Coronado, CA (US); Kim Vanderlinden, Coronado, CA (US); Carla Janie Rosewall, Coronado, CA (US)

(21) Appl. No.: 19/241,092

(22) Filed: Jun. 17, 2025

Related U.S. Application Data
(60) Provisional application No. 63/660,676, filed on Jun. 17, 2024.

(51) Int. Cl. A61K 31/6615; A61K 9/00; A61K 9/06; A61K 31/047; A61K 36/899; A61K 47/10; A61K 47/14

(52) U.S. Cl. CPC A61K 31/6615; A61K 9/0014; A61K 9/06; A61K 31/047; A61K 36/899; A61K 47/10; A61K 47/14

(57) ABSTRACT

The invention is implemented into the category of transdermal topical agents for use in regulating the dilation of blood vessels. The IP-6, Inositol, and colloidal oatmeal Nerve Relaxing Transdermal Topical Cream, Gel, or Ointment for Raynaud's Syndrome is the first topical cream containing any or all of the following: IP-6, inositol, and colloidal oatmeal, with IP-6 as the carrier agent. The present invention will allow for the decrease in the time to full clinical effect, eliminate having to wear a patch of paper over the application site, and eliminate the problem of skin irritation due to the IP-6 delivering the colloidal oatmeal to the application area to provide extra soothing and moisturizing properties. The present invention is specifically and uniquely designed for ease of use and quick action of full clinical effect with the relaxation of nerve endings that lead to dilation of extremely constricted blood vessels in mind.

### RELATED APPLICATION

This application claims the benefit of priority to U.S. Provisional Patent Application for “IP-6 and Inositol Nerve Relaxing Transdermal Topical Cream, Gel, or Ointment for Raynaud's Syndrome”, Ser. No. 63/660,676, filed on Jun. 17, 2024, and currently co-pending. The above-mentioned related application is incorporated fully herein by this reference.

### FIELD OF THE INVENTION

The present invention relates generally to the novel composition and transdermal delivery method of substances for the relief of symptoms of Raynaud's syndrome. The present invention is particularly a composition comprised of any or all of the following: IP-6, inositol, and colloidal oatmeal. The present invention also relates to pharmaceutical compositions and combined preparations of IP-6, inositol, and colloidal oatmeal, along with a dosage regime for their administration. The present invention is also particularly, but not exclusively, useful as a treatment for instances and occurrences of the extreme blood vessel constriction associated with Raynaud's syndrome by reducing the intensity of the firing of nerve endings that constrict the blood vessels, and thereby restoring the proper dilation of the blood vessels in the affected areas (i.e., fingers, toes, nose, etc.). The present invention is also particularly, but not exclusively, useful as a treatment for instances and occurrences of the numbness, prickling, tingling, pain, and muscle weakness in feet or hands due to peripheral neuropathy by relaxing nerve endings in the application area leading to an immediate reduction in the intensity of symptoms of discomfort associated with nerve damage. The present invention is also particularly, but not exclusively, useful as a treatment for instances and occurrences of neurogenic erectile dysfunction by relaxing the nerve endings in the application area.

### BACKGROUND OF THE INVENTION

Raynaud's syndrome, also known as Raynaud's phenomenon, is a medical condition where certain areas of the body, typically the fingers and toes, but sometimes also the ears or nose, will have their blood vessels narrow excessively in response to cold temperature, stress, stimulants (i.e., caffeine; nicotine), medications, or even exercise. The excessive narrowing of the blood vessels leads to the affected digits feeling very cold, a loss of sensation in the digits, and skin discoloration of the digits. The skin discoloration has three stages, first of which is when the fingers and/or toes turn white (pallor) from the decreased blood flow, second, they turn blue (cyanosis) from the decreased oxygen, and finally red (reperfusion) when the oxygenated blood returns to the digits after the restriction has ceased. In severe cases of Raynaud's syndrome, the decreased blood circulation can lead to tissue damage in the digits such as ulcers or pitting.

Raynaud's syndrome is separated into two categories, primary and secondary. Primary Raynaud's is solely Raynaud's and is triggered by cold temperature, stress, stimulants, medications, or exercise. Whereas secondary Raynaud's may be associated with underlying diseases such as lupus, scleroderma, rheumatoid arthritis, or atherosclerosis.

Historically, Raynaud's syndrome has been treated with different methods having minimal levels of success, including: calcium channel blockers; alpha blockers; phosphodiesterase inhibitors; pulmonary hypertension medications; Botox injections; selective serotonin reuptake inhibitors; statins; and transdermal topical agents.

Currently, the most common transdermal topical agent for dealing with Raynaud's syndrome is topical nitroglycerin, a substance which is usually used to prevent angina, but also has a venodilating and vasodilating effect on the applied area that decreases the arterial blood pressure in the area, and the relief lasts for 7-10 hours. The problems with this option include having to accurately measure the dose, rotate sites of application to prevent irritation of the skin, and it is advised to not rub the ointment into the skin which means that there must be a piece of paper or patch taped on top of the application site to prevent the ointment from going onto clothes or rubbing itself into the skin. The time to onset of action for nitroglycerin ointment is between 9-11 minutes with the time to full clinical effect being 14-20 minutes.

There is some early-stage, preliminary research that food and dietary supplements such as vitamin B complex may also aid in the relief of the symptoms of Raynaud's syndrome, specifically vitamin B3, niacin, and vitamin B8, inositol. One of these B vitamins, inositol, commonly known as vitamin B8, is a sugar made in the body and found in food, with its most common form being myo-inositol has also been shown to aid with blood pressure issues.

IP-6, also known as phytate, inositol hexaphosphate, and inositol hexakisphosphate, has known antioxidant properties and is sometimes used in combination with myo-inositol to reduce chemotherapy side effects, however, it has been revealed through case studies of the present invention that IP-6 alone or in combination with inositol and colloidal oatmeal may also offer other health benefits, specifically relieving the symptoms of Raynaud's syndrome. IP-6 can penetrate the epidermis or outer layer of the skin to reach the dermis and hypodermis. It was found that IP-6 is easily absorbed through the skin by using either gel or cream.

Colloidal oatmeal can be used to relax the nerves and sooth the nerve endings where it is applied to. The IP-6 acts as the carrier to deliver IP-6, inositol, and colloidal oatmeal into the dermis and hypodermis of the affected area. IP-6 penetrates the epidermis or outer layer of the skin to reach the dermis and hypodermis whilst carrying the inositol and colloidal oatmeal into the same area. This allows the affected area to receive the benefits of the IP-6, inositol, as well as those of the colloidal oatmeal. The colloidal oatmeal, inositol, and IP-6 work synergistically to relax the nerve endings and regulate their activation and subsequent firing.

There can be difficulty obtaining some of the products and medications used to address the extreme constriction of blood vessels, the time taken to reach full clinical effect, as well as ease of use issues and irritation of the skin associated with the use of topical nitroglycerin. A readily available transdermal topical agent using IP-6, inositol, and colloidal oatmeal will address some of these issues. The IP-6 being utilized as the carrier agent will decrease the time taken to reach full clinical effect, will not irritate the skin, as well as increase the ease of use.

Peripheral neuropathy occurs when the nerves located outside the brain and spinal cord (the peripheral nerves) become damaged and leads to numbness, prickling, tingling, pain, and muscle weakness in feet or hands. The most common cause is diabetes, i.e., diabetic neuropathy.

Historically the peripheral neuropathy caused by diabetes may be controlled by regulating blood sugar levels, blood pressure, cholesterol levels, and weight to keep the nerve damage from worsening.

Currently the treatment options for peripheral neuropathy consists of lifestyle changes such as proper nutrition to regulate blood sugar levels, blood pressure, cholesterol levels and weight; using medications to achieve optimal diabetic control such as Ibuprofen, Venlafaxine, Nortriptyline, Gabapentin, Pregabalin, glucagon-like peptide-1 (GLP-1), such as semaglutide; and therapies such as Transcutaneous Electrical Nerve Stimulation (TENS) and Plasmapheresis.

Because of the difficulty of obtaining some of the current medications, and lack of access to the therapies used to address the nerve sensitivity of peripheral neuropathy. A readily available transdermal topical agent is IP-6, inositol, and colloidal oatmeal. The IP-6 being utilized as the carrier agent, will allow for soothing and relaxation of the peripheral nerve endings with a decrease in time to reach full clinical effect.

Erectile dysfunction is a form of sexual dysfunction in males that is characterized by the persistent or recurring inability to achieve or maintain a penile erection with sufficient rigidity and duration for satisfactory sexual activity. Causes include physical conditions including but not limited to: neurological issues, trauma to the penis or spinal cord, diseases of the penis, diseases of the heart or blood vessels (atherosclerosis, myocardial infarction), high blood pressure, diabetes, metabolic syndrome; and psychological conditions including but not limited to depression, anxiety, and stress. According to the CDC, men with diabetes are three times more likely to have erectile dysfunction.

Currently erectile dysfunction has many options available for treatment, such as medications like Sildenafil and Tadalafil, as well as topical nitroglycerin that enhance nitric oxide effects; penile self-injections of medications like Papaverine and Alprostadil; surgeries to implant devices into the penile tissue; chelation therapy for atherosclerosis; and hormone replacement therapy. Specifically, current treatments for erectile dysfunction related to diabetes include keeping blood sugar levels low to avoid or lessen nerve and blood vessel damage, managing blood pressure and cholesterol levels.

Because of the difficulty of obtaining some of the medications for erectile dysfunction, and lack of access to the therapies used to address the local blood circulation issues that may cause one to experience erectile dysfunction. A readily available transdermal topical agent is IP-6, inositol, and colloidal oatmeal. The IP-6 being utilized as the carrier agent will decrease the time taken to reach full clinical effect and will not irritate the skin.

In view of the above, it would be advantageous to provide an easy-to-use transdermal topical cream with a fast-acting carrier that provides quick acting and long-lasting relief to extremely constricted blood vessels, damaged nerves, and areas lacking local blood flow. Incidentally, the same transdermal topical cream will be able to relax and soothe the nerve endings in the application area. Thus, improving efficiency and speed of relief when using a transdermal topical agent for treating instances of Raynaud's syndrome, peripheral neuropathy, and erectile dysfunction.

### SUMMARY OF THE INVENTION

Provided herein are compositions, and application methods for transdermal delivery of substances using a topical nerve ending relaxing cream, gel, or ointment for treatment of Raynaud's syndrome, peripheral neuropathy, and erectile dysfunction symptoms and conditions. In which the salt form of IP-6 includes, but are not restricted to calcium-magnesium, sodium, potassium, calcium, and magnesium salts. In one embodiment that includes IP-6, inositol, and colloidal oatmeal, the IP-6 is utilized as the carrier agent.

The compositions described herein overcome multiple problems that arise from treating Raynaud's syndrome with the current topical therapeutic compositions. The compositions also may be used to treat the symptoms of peripheral neuropathy, and erectile dysfunction. For example, the compositions are chemically and physically stable in storage conditions of 33° F. to 85° F./30%-70% relative humidity (RH), and the compositions achieve a symptom resolving relevant bioavailability of the active ingredients, IP-6, inositol, and colloidal oatmeal in the target dermal area of application as observed in case studies.

The compositions of the present invention are especially useful for treating individuals suffering from Raynaud's syndrome symptoms of extremely restricted blood vessels in the digits by reducing the intensity of the firing of nerve endings of the blood vessels resulting in widening of the blood vessels in the digits through vasodilation.

The compositions of the present invention are incidentally useful for treating individuals suffering from the symptoms of peripheral neuropathy, more specifically, diabetic neuropathy due to the fast-acting soothing and relaxing effects that the IP-6, inositol, and colloidal oatmeal combination have on the damaged nerve endings.

The compositions of the present invention are incidentally useful for treating individuals suffering from neurogenic erectile dysfunction due to the composition relaxing the nerve endings in the application area.

The results and properties achieved by the present invention are due to the selection of the ingredients and their relative amounts. More particularly the transdermal topical cream of the present invention contains, inositol, IP-6, and colloidal oatmeal. In a preferred embodiment, calcium magnesium inositol hexakiphosphate (CaMgIP6) ranging from 1-15% on a weight to weight basis of the composition, inositol ranging from 1-15% on a weight to weight basis of the composition, colloidal oatmeal ranging from 0.1-15% on a weight to weight basis of the composition, aqua (deionized water), glycerin, glyceryl stearate, phenoxyethanol, and zemea (corn) propanediol; this embodiment may be used to treat Raynaud's syndrome, peripheral neuropathy, and erectile dysfunction symptoms.

The compositions of the present invention use IP-6 as the carrier agent and will allow for the decrease in the time to full clinical effect. The IP-6 acts as a penetration enhancer for dermal application in an amount of about 1%-15% on a weight-to-weight basis of the composition. The time for full clinical effect when using IP-6 is 5- 30 minutes. Moreover, the use of IP-6 as the carrier agent will avoid having to wear a patch of paper over the application site, the problem of not being able to rub in the cream, as well as the problem of skin irritation due to the IP-6 delivering the colloidal oatmeal to the application area to provide extra soothing and moisturizing properties. In this embodiment the preferred salt form of IP-6 will be the calcium magnesium inositol hexakiphosphate salt form, however other pharmacologically acceptable salt forms of IP-6 including, but not restricted to: calcium-magnesium, sodium, potassium, calcium, and magnesium salts may also be used.

It is one of the main objects of the present invention to provide a highly stable transdermal topical skin cream that has vasodilating properties with an Inositol to IP-6 ratio ranging between a 1:15 to 15:1 molar ratio of Inositol:IP-6.

It is also one of the main objects of this invention to provide for a method of use of the topical cream that includes dispensing a 5-20 mm sized dollop of the cream onto the affected area, rubbing the dollop into the skin of the affected area(s) until there is a thin film coating the area(s), waiting 5 - 30 minutes for the full effect of the transported substances, the user may wash off the film afterwards if they so choose. The ideal daily frequency of application would be one to two times daily with a third application based on the severity of the condition.

It is another object of the present invention to provide composition that is quick acting to achieve the time of full clinical effect.

It is still another object of this invention to provide such a composition that is simple to use and may be rubbed into the affected areas without irritation of the skin.

Further objects of the invention will be brought out into the following part of the specification wherein detailed description is for the purpose of fully disclosing the invention without placing limitations thereon.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a flow diagram illustrating the steps executed in using the present invention for Raynaud's Syndrome/Peripheral Neuropathy/Erectile Dysfunction, and intervals between application;

FIG. 2 is a two-dimensional front view of the chemical structure of inositol C6H12O6 used in the preferred embodiment;

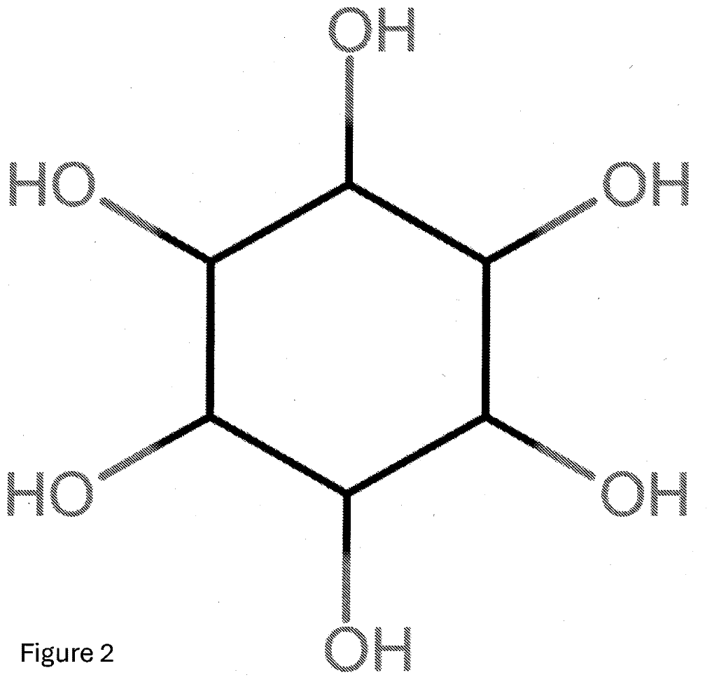

FIG. 3 is a two-dimensional front view of the chemical structure of calcium magnesium inositol hexakiphosphate C6H6CaMgO24P6-8 used in the preferred embodiment.

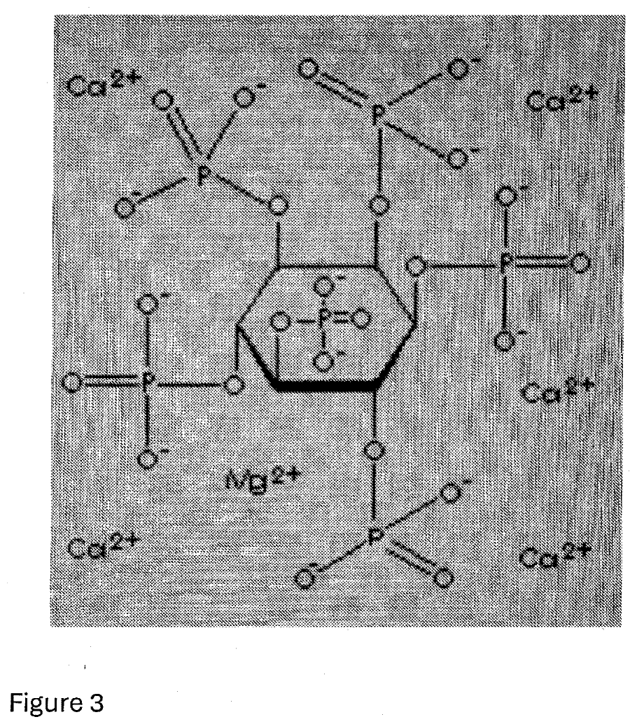

### DETAILED DESCRIPTION

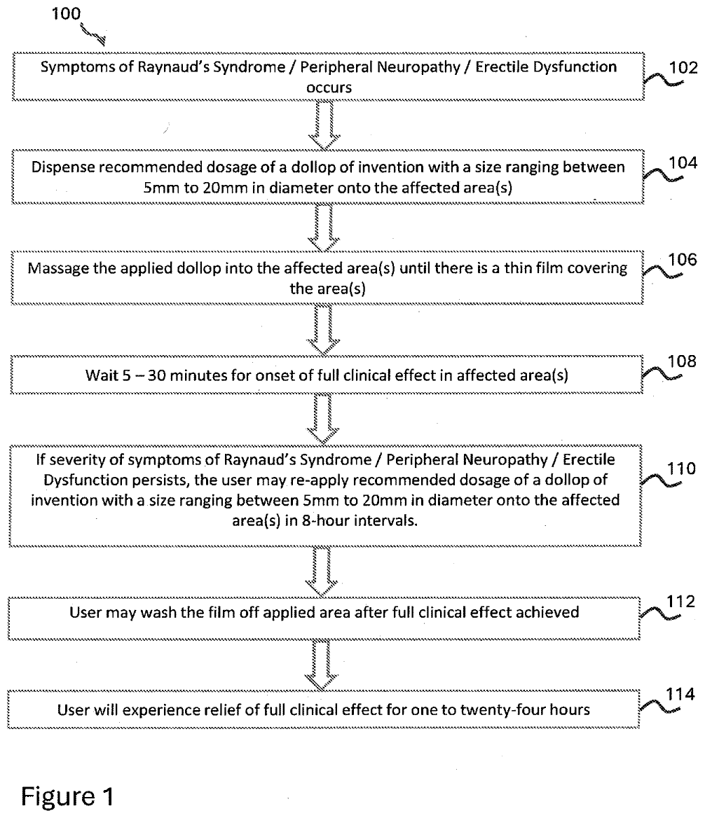

Referring initially to FIG. 1, the method of use of the IP-6 and Inositol Transdermal Topical Cream is illustrated by a flow diagram generally designated 100. When symptoms of Raynaud's Syndrome/Peripheral Neuropathy/Erectile Dysfunction occur 102, the user should dispense the recommended dosage of a dollop of the invention with a size ranging between 5 mm to 20 mm in diameter onto the affected area(s) 104, and massage the applied dollop into the affected area(s) until there is a thin film covering the affected area(s) 106. The user should wait 5 - 30 minutes for the onset of the full clinical effect in the affected area(s) 108. If the severity of the symptoms of Raynaud's Syndrome/Peripheral Neuropathy/Erectile Dysfunction persists, the user may re-apply the recommended dosage in the size stated in step 104. After the full clinical effect is achieved the user may wash the film off the applied area 112. The user will experience relief of the symptoms of Raynaud's Syndrome/Peripheral Neuropathy/Erectile Dysfunction for one to twenty-four hours 114.

The following examples are given as specific illustrations of the invention. It should be understood, however, that the invention is not limited to the specific details set forth therein. In the following examples, all percentages not included in the name of the component are by weight of the total composition.

### Example I

One example of the present invention includes the following ingredient list: colloidal oatmeal base cream 0.1% on a weight to weight basis of the composition, calcium magnesium inositol hexakiphosphate (CaMgIP6) ranging from 1-15% on a weight to weight basis of the composition, inositol ranging from 1-15% on a weight to weight basis of the composition, aqua (deionized water), glycerin, glyceryl stearate, phenoxyethanol, zemea (corn) propanediol.

### Example I(a)

A specific composition of the ingredient list may include: colloidal oatmeal base cream 0.1% on a weight to weight basis of the composition, calcium magnesium inositol hexakiphosphate (CaMgIP6) 1% on a weight to weight basis of the composition, inositol 1% on a weight to weight basis of the composition, aqua (deionized water, glycerin, glyceryl stearate, phenoxyethanol, zemea (corn) propanediol.

### Example I(b)

A specific composition of the ingredient list may include: colloidal oatmeal base cream 0.1% on a weight to weight basis of the composition, calcium magnesium inositol hexakiphosphate (CaMgIP6) 5% on a weight to weight basis of the composition, inositol 5% on a weight to weight basis of the composition, aqua (deionized water), glycerin, glyceryl stearate, phenoxyethanol, zemea (corn) propanediol.

### Example I(c)

A specific composition of the ingredient list may include: colloidal oatmeal base cream 0.1% on a weight to weight basis of the composition, calcium magnesium inositol hexakiphosphate (CaMgIP6) 10% on a weight to weight basis of the composition, inositol 10% on a weight to weight basis of the composition, aqua (deionized water, glycerin, glyceryl stearate, phenoxyethanol, zemea (corn) propanediol.

### Example I(d)

A specific composition of the ingredient list may include: colloidal oatmeal base cream 0.1% on a weight to weight basis of the composition, calcium magnesium inositol hexakiphosphate (CaMgIP6) 15% on a weight to weight basis of the composition, inositol 15% on a weight to weight basis of the composition, aqua (deionized water), glycerin, glyceryl stearate, phenoxyethanol, zemea (corn) propanediol.

### Example II

Another example of the present invention includes the following ingredient list: colloidal oatmeal base cream 0.5% on a weight to weight basis of the composition, calcium magnesium inositol hexakiphosphate (CaMgIP6) ranging from 1-15% on a weight to weight basis of the composition, inositol 1-15% on a weight to weight basis of the composition, aqua (deionized water), glycerin, glyceryl stearate, phenoxyethanol, zemea (corn) propanediol.

### Example II(a)

A specific composition of the ingredient list may include: colloidal oatmeal base cream 0.5% on a weight to weight basis of the composition, calcium magnesium inositol hexakiphosphate (CaMgIP6) 1% on a weight to weight basis of the composition, inositol 1% on a weight to weight basis of the composition, aqua (deionized water), glycerin, glyceryl stearate, phenoxyethanol, zemea (corn) propanediol.

### Example II(b)

A specific composition of the ingredient list may include: colloidal oatmeal base cream 0.5% on a weight to weight basis of the composition, calcium magnesium inositol hexakiphosphate (CaMgIP6) 5% on a weight to weight basis of the composition, inositol 5% on a weight to weight basis of the composition, aqua (deionized water), glycerin, glyceryl stearate, phenoxyethanol, zemea (corn) propanediol.

### Example II(c)

A specific composition of the ingredient list may include: colloidal oatmeal base cream 0.5% on a weight to weight basis of the composition, calcium magnesium inositol hexakiphosphate (CaMgIP6) 10% on a weight to weight basis of the composition, inositol 10% on a weight to weight basis of the composition, aqua (deionized water), glycerin, glyceryl stearate, phenoxyethanol, zemea (corn) propanediol.

### Example II(d)

A specific composition of the ingredient list may include: colloidal oatmeal base cream 0.5% on a weight to weight basis of the composition, calcium magnesium inositol hexakiphosphate (CaMgIP6) 15% on a weight to weight basis of the composition, inositol 15% on a weight to weight basis of the composition, aqua (deionized water), glycerin, glyceryl stearate, phenoxyethanol, zemea (corn) propanediol.

### Example III

Yet another example of the present invention includes the following ingredient list: colloidal oatmeal base cream 1% on a weight to weight basis of the composition, calcium magnesium inositol hexakiphosphate (CaMgIP6) ranging from 1-15% on a weight to weight basis of the composition, inositol ranging from 1-15% on a weight to weight basis of the composition, aqua (deionized water), glycerin, glyceryl stearate, phenoxyethanol, zemea (corn) propanediol.

### Example III(a)

A specific composition of the ingredient list may include: colloidal oatmeal base cream 1% on a weight to weight basis of the composition, calcium magnesium inositol hexakiphosphate (CaMgIP6) 1% on a weight to weight basis of the composition, inositol 1% on a weight to weight basis of the composition, aqua (deionized water), glycerin, glyceryl stearate, phenoxyethanol, zemea (corn) propanediol.

### Example III(b)

A specific composition of the ingredient list may include: colloidal oatmeal base cream 1% on a weight to weight basis of the composition, calcium magnesium inositol hexakiphosphate (CaMgIP6) 5% on a weight to weight basis of the composition, inositol 5% on a weight to weight basis of the composition, aqua (deionized water), glycerin, glyceryl stearate, phenoxyethanol, zemea (corn) propanediol.

### Example III(c)

A specific composition of the ingredient list may include: colloidal oatmeal base cream 1% on a weight to weight basis of the composition, calcium magnesium inositol hexakiphosphate (CaMgIP6) 10% on a weight to weight basis of the composition, inositol 10% on a weight to weight basis of the composition, aqua (deionized water), glycerin, glyceryl stearate, phenoxyethanol, zemea (corn) propanediol.

### Example III(d)

A specific composition of the ingredient list may include: colloidal oatmeal base cream 1% on a weight to weight basis of the composition, calcium magnesium inositol hexakiphosphate (CaMgIP6) 15% on a weight to weight basis of the composition, inositol 15% on a weight to weight basis of the composition, aqua (deionized water), glycerin, glyceryl stearate, phenoxyethanol, zemea (corn) propanediol.

### Example IV

Yet another example of the present invention includes the following ingredient list: colloidal oatmeal base cream 2% on a weight to weight basis of the composition, calcium magnesium inositol hexakiphosphate (CaMgIP6) ranging from 1-15% on a weight to weight basis of the composition, inositol ranging from 1-15% on a weight to weight basis of the composition, aqua (deionized water), glycerin, glyceryl stearate, phenoxyethanol, zemea (corn) propanediol.

### Example IV(a)

A specific composition of the ingredient list may include: colloidal oatmeal base cream 2% on a weight to weight basis of the composition, calcium magnesium inositol hexakiphosphate (CaMgIP6) 1% on a weight to weight basis of the composition, inositol 1% on a weight to weight basis of the composition, aqua (deionized water), glycerin, glyceryl stearate, phenoxyethanol, zemea (corn) propanediol.

### Example IV(b)

A specific composition of the ingredient list may include: colloidal oatmeal base cream 2% on a weight to weight basis of the composition, calcium magnesium inositol hexakiphosphate (CaMgIP6) 5% on a weight to weight basis of the composition, inositol 5% on a weight to weight basis of the composition, aqua (deionized water), glycerin, glyceryl stearate, phenoxyethanol, zemea (corn) propanediol.

### Example IV(c)

A specific composition of the ingredient list may include: colloidal oatmeal base cream 2% on a weight to weight basis of the composition, calcium magnesium inositol hexakiphosphate (CaMgIP6) 10% on a weight to weight basis of the composition, inositol 10% on a weight to weight basis of the composition, aqua (deionized water), glycerin, glyceryl stearate, phenoxyethanol, zemea (corn) propanediol.

### Example IV(d)

A specific composition of the ingredient list may include: colloidal oatmeal base cream 2% on a weight to weight basis of the composition, calcium magnesium inositol hexakiphosphate (CaMgIP6) 15% on a weight to weight basis of the composition, inositol 15% on a weight to weight basis of the composition, aqua (deionized water), glycerin, glyceryl stearate, phenoxyethanol, zemea (corn) propanediol.

### Example V

Yet another example of the present invention includes the following ingredient list: colloidal oatmeal base cream 3% on a weight to weight basis of the composition, calcium magnesium inositol hexakiphosphate (CaMgIP6) ranging from 1-15% on a weight to weight basis of the composition, inositol ranging from 1-15% on a weight to weight basis of the composition, aqua (deionized water), glycerin, glyceryl stearate, phenoxyethanol, zemea (corn) propanediol.

### Example V(a)

A specific composition of the ingredient list may include: colloidal oatmeal base cream 3% on a weight to weight basis of the composition, calcium magnesium inositol hexakiphosphate (CaMgIP6) 1% on a weight to weight basis of the composition, inositol 1% on a weight to weight basis of the composition, aqua (deionized water), glycerin, glyceryl stearate, phenoxyethanol, zemea (corn) propanediol.

### Example V(b)

A specific composition of the ingredient list may include: colloidal oatmeal base cream 3% on a weight to weight basis of the composition, calcium magnesium inositol hexakiphosphate (CaMgIP6) 5% on a weight to weight basis of the composition, inositol 5% on a weight to weight basis of the composition, aqua (deionized water), glycerin, glyceryl stearate, phenoxyethanol, zemea (corn) propanediol.

### Example V(c)

A specific composition of the ingredient list may include: colloidal oatmeal base cream 3% on a weight to weight basis of the composition, calcium magnesium inositol hexakiphosphate (CaMgIP6) 10% on a weight to weight basis of the composition, inositol 10% on a weight to weight basis of the composition, aqua (deionized water), glycerin, glyceryl stearate, phenoxyethanol, zemea (corn) propanediol.

### Example V(d)

A specific composition of the ingredient list may include: colloidal oatmeal base cream 3% on a weight to weight basis of the composition, calcium magnesium inositol hexakiphosphate (CaMgIP6) 15% on a weight to weight basis of the composition, inositol 15% on a weight to weight basis of the composition, aqua (deionized water), glycerin, glyceryl stearate, phenoxyethanol, zemea (corn) propanediol.

### Example VI

Yet another example of the present invention includes the following ingredient list: colloidal oatmeal base cream 6% on a weight to weight basis of the composition, calcium magnesium inositol hexakiphosphate (CaMgIP6) ranging from 1-15% on a weight to weight basis of the composition, inositol ranging from 1-15% on a weight to weight basis of the composition, aqua (deionized water), glycerin, glyceryl stearate, phenoxyethanol, zemea (corn) propanediol.

### Example VI(a)

A specific composition of the ingredient list may include: colloidal oatmeal base cream 6% on a weight to weight basis of the composition, calcium magnesium inositol hexakiphosphate (CaMgIP6) 1% on a weight to weight basis of the composition, inositol 1% on a weight to weight basis of the composition, aqua (deionized water), glycerin, glyceryl stearate, phenoxyethanol, zemea (corn) propanediol.

### Example VI(b)

A specific composition of the ingredient list may include: colloidal oatmeal base cream 6% on a weight to weight basis of the composition, calcium magnesium inositol hexakiphosphate (CaMgIP6) 5% on a weight to weight basis of the composition, inositol 5% on a weight to weight basis of the composition, aqua (deionized water), glycerin, glyceryl stearate, phenoxyethanol, zemea (corn) propanediol.

### Example VI(c)

A specific composition of the ingredient list may include: colloidal oatmeal base cream 6% on a weight to weight basis of the composition, calcium magnesium inositol hexakiphosphate (CaMgIP6) 10% on a weight to weight basis of the composition, inositol 10% on a weight to weight basis of the composition, aqua (deionized water), glycerin, glyceryl stearate, phenoxyethanol, zemea (corn) propanediol.

### Example VI(d)

A specific composition of the ingredient list may include: colloidal oatmeal base cream 6% on a weight to weight basis of the composition, calcium magnesium inositol hexakiphosphate (CaMgIP6) 15% on a weight to weight basis of the composition, inositol 15% on a weight to weight basis of the composition, aqua (deionized water), glycerin, glyceryl stearate, phenoxyethanol, zemea (corn) propanediol.

### Example VII

Yet another example of the present invention includes the following ingredient list: colloidal oatmeal base cream 10% on a weight to weight basis of the composition, calcium magnesium inositol hexakiphosphate (CaMgIP6) ranging from 1-15% on a weight to weight basis of the composition, inositol 1-15% on a weight to weight basis of the composition, aqua (deionized water), glycerin, glyceryl stearate, phenoxyethanol, zemea (corn) propanediol.

### Example VII(a)

A specific composition of the ingredient list may include: colloidal oatmeal base cream 10% on a weight to weight basis of the composition, calcium magnesium inositol hexakiphosphate (CaMgIP6) 1% on a weight to weight basis of the composition, inositol 1% on a weight to weight basis of the composition, aqua (deionized water), glycerin, glyceryl stearate, phenoxyethanol, zemea (corn) propanediol.

### Example VII(b)

A specific composition of the ingredient list may include: colloidal oatmeal base cream 10% on a weight to weight basis of the composition, calcium magnesium inositol hexakiphosphate (CaMgIP6) 5% on a weight to weight basis of the composition, inositol 5% on a weight to weight basis of the composition, aqua (deionized water), glycerin, glyceryl stearate, phenoxyethanol, zemea (corn) propanediol.

### Example VII(c)

A specific composition of the ingredient list may include: colloidal oatmeal base cream 10% on a weight to weight basis of the composition, calcium magnesium inositol hexakiphosphate (CaMgIP6) 10% on a weight to weight basis of the composition, inositol 10% on a weight to weight basis of the composition, aqua (deionized water), glycerin, glyceryl stearate, phenoxyethanol, zemea (corn) propanediol.

### Example VII(d)

A specific composition of the ingredient list may include: colloidal oatmeal base cream 10% on a weight to weight basis of the composition, calcium magnesium inositol hexakiphosphate (CaMgIP6) 15% on a weight to weight basis of the composition, inositol 15% on a weight to weight basis of the composition, aqua (deionized water), glycerin, glyceryl stearate, phenoxyethanol, zemea (corn) propanediol.

### Example VIII

Yet another example of the present invention includes the following ingredient list: colloidal oatmeal base cream 15% on a weight to weight basis of the composition, calcium magnesium inositol hexakiphosphate (CaMgIP6) ranging from 1-15% on a weight to weight basis of the composition, inositol ranging from 1-15% on a weight to weight basis of the composition, aqua (deionized water), glycerin, glyceryl stearate, phenoxyethanol, zemea (corn) propanediol.

### Example VIII(a)

A specific composition of the ingredient list may include: colloidal oatmeal base cream 15% on a weight to weight basis of the composition, calcium magnesium inositol hexakiphosphate (CaMgIP6) 1% on a weight to weight basis of the composition, inositol 1% on a weight to weight basis of the composition, aqua (deionized water), glycerin, glyceryl stearate, phenoxyethanol, zemea (corn) propanediol.

### Example VIII(b)

A specific composition of the ingredient list may include: colloidal oatmeal base cream 15% on a weight to weight basis of the composition, calcium magnesium inositol hexakiphosphate (CaMgIP6) 5% on a weight to weight basis of the composition, inositol 5% on a weight to weight basis of the composition, aqua (deionized water), glycerin, glyceryl stearate, phenoxyethanol, zemea (corn) propanediol.

### Example VIII(c)

A specific composition of the ingredient list may include: colloidal oatmeal base cream 15% on a weight to weight basis of the composition, calcium magnesium inositol hexakiphosphate (CaMgIP6) 10% on a weight to weight basis of the composition, inositol 10% on a weight to weight basis of the composition, aqua (deionized water), glycerin, glyceryl stearate, phenoxyethanol, zemea (corn) propanediol.

### Example VIII(d)

A specific composition of the ingredient list may include: colloidal oatmeal base cream 15% on a weight to weight basis of the composition, calcium magnesium inositol hexakiphosphate (CaMgIP6) 15% on a weight to weight basis of the composition, inositol 15% on a weight to weight basis of the composition, aqua (deionized water), glycerin, glyceryl stearate, phenoxyethanol, zemea (corn) propanediol.

The above examples are a sample set of the multiple combinations of the composition that may be manufactured.

In various embodiments, the present invention will utilize inositol C6H12O6.

In various embodiments, the present invention will utilize calcium magnesium inositol hexakiphosphate C6H6CaMgO24P6-8, but may use any pharmacologically acceptable salt form of IP-6 including, but not restricted to: calcium-magnesium, sodium, potassium, calcium, and magnesium salts.

In various embodiments, the present invention will utilize colloidal oatmeal.

In various embodiments, the present invention will utilize aqua (deionized water).

In various embodiments, the present invention will utilize glycerin.

In various embodiments, the present invention will utilize glyceryl stearate.

In various embodiments, the present invention will utilize phenoxyethanol.

In various embodiments, the present invention will utilize zemea (corn) propanediol.

In various embodiments, the present invention will utilize a dosage of a dollop with a size ranging between 5 mm to 20 mm in diameter.

In various embodiments, the present invention will be applied to the affected area(s) with the preferred dosage and rubbed into the affected area(s) until there is a thin film covering the area(s).

In various embodiments, the present invention will have the onset of its full clinical effect within 5-30 minutes.

In various embodiments, the present invention will have a full clinical effect ranging between 1 to 24 hours.

In various embodiments, the present invention may be used in intervals of 8 to 24 hours.

In various embodiments the present invention will penetrate and be absorbed into the epidermis, dermis and hypodermis layers of the skin.

In various embodiments, the present invention will relax and soothe the nerves in the application area.

In various embodiments, the present invention may be used to improve issues related to neurogenic erectile dysfunction through relaxation of the nerve endings.

In various embodiments, the present invention may be used to improve issues related to Raynaud's Syndrome through relaxation of the nerve endings.

In various embodiments, the present invention may be used to improve issues related to and diabetic neuropathy by relaxing nerve endings in the application area leading to an immediate reduction in the intensity of symptoms of discomfort associated with nerve damage.

In various embodiments, the present invention may be used to improve issues related to hair loss.

In various embodiments, the present invention may improve blood glucose levels.

In various embodiments, the present invention may be used to improve issues related to platelet aggregation through increasing blood flow and decreasing blood clotting.

In various embodiments, the present invention may be used to help in the management of bone density through its restorative properties, thus avoiding decreases in bone density.

While there have been shown what are presently considered to be preferred embodiments of the present invention, it will be apparent to those skilled in the art that various changes and modifications can be made herein without departing from the scope and spirit of the invention.

What is claimed is:

1. A composition, formulated for topical administration, useful for the treatment of Raynaud's syndrome, peripheral neuropathy, and erectile dysfunction symptoms, comprising: Calcium Magnesium Inositol Hexakiphosphate (CaMgIP6), Inositol (C6H12O6), colloidal oatmeal, aqua (deionized water), glycerin, glyceryl stearate, phenoxyethanol, and zemea (corn) propanediol.

2. The composition of claim 1 wherein the Calcium Magnesium Inositol Hexakiphosphate (CaMgIP6) is in a concentration ranging from 1-15% on a weight to weight basis of the composition.

3. The composition of claim 1 wherein the Inositol (C6H12O6) is in a concentration ranging from 1-15% on a weight to weight basis of the composition.

4. The composition of claim 1 wherein the colloidal oatmeal is in a concentration ranging from 0.1-15% on a weight to weight basis of the composition.

5. The composition of claim 1 wherein the carrier agent is the IP-6.

6. The composition of claim 1 wherein the salt form of IP-6 may be Calcium Magnesium Inositol Hexakiphosphate (CaMgIP6), or any other pharmacologically acceptable salt form of IP-6.

7. The composition of claim 1 wherein it may be in either a cream, gel, or ointment form.

8. The composition of claim 1 wherein the application of said composition relaxes and soothes the nerves in the application area.

9. The composition of claim 1 wherein the application of said composition may improve issues related to hair loss.

10. The composition of claim 1 wherein the application of said composition may improve blood glucose levels.

11. The composition of claim 1 wherein the application of said composition may improve issues related to platelet aggregation.

12. The composition of claim 1 wherein the application of said composition may improve issues related to bone density loss.

13. The composition of claim 1 wherein the time for full clinical effect is achieved within a range of five to thirty minutes.

14. The composition of claim 1 wherein the full clinical effect is maintained for a range of one to twenty-four hours.

15. A method of treating Raynaud's Syndrome, Peripheral Neuropathy, and Erectile Dysfunction, comprising: topically administering a therapeutically effective amount of the composition of claim 1 to the affected area(s) of a patient.

16. The method of claim 15, wherein the composition's ingredients, Calcium Magnesium Inositol Hexakiphosphate (CaMgIP6), Inositol (C6H12O6), and colloidal oatmeal are in concentrations ranging from 1-15%; 1-15%; and 0.1-15% on a weight to weight basis of the composition, respectively.

17. The method of claim 15 wherein the amount of the composition used is a dollop size ranging between 5 mm to 20 mm in diameter that is massaged into the affected area(s) of the patient.

18. The method of claim 15 wherein the patient waits five to thirty minutes to feel the full clinical effect of the composition in the affected area(s).

19. The method of claim 15 wherein the patient experiences the full clinical effect for a range of one to twenty-four hours.

20. The method of claim 15 wherein the patient may reapply a dollop size ranging between 5 mm to 20 mm in diameter in eight-hour intervals.
